(19) United States
(12) Patent Application Publication
(10) Pub. No.: US 2025/0091988 A1
(43) Pub. Date: Mar. 20, 2025

(54) MULTIFUNCTIONAL GROUP METAL CORROSION INHIBITOR, PREPARATION METHOD THEREFOR, AND APPLICATION THEREOF

(71) Applicant: TAN KAH KEE INNOVATION LABORATORY, Xiamen (CN)

(72) Inventors: Aiqiang Zhang, Xiamen (CN); Ting Chen, Xiamen (CN); Mark Neisser, Xiamen (CN); Songsong Luo, Xiamen (CN); Congjie Lu, Xiamen (CN); Weishun Lai, Xiamen (CN)

(21) Appl. No.: 18/729,652

(22) PCT Filed: Jan. 17, 2022
(86) PCT No.: PCT/CN2022/072275

§ 371 Date: Jul. 17, 2024

(51) Int. Cl. C07C 323/34; C07C 319/12; C07D 277/66; C11D 3/00; C11D 3/34

(52) U.S. Cl. CPC C07C 323/34; C07C 319/12; C07D 277/66; C11D 3/0073; C11D 3/349; C07C 2601/16; C11D 2111/22

(57) ABSTRACT

Provided is a multifunctional group metal corrosion inhibitor, as shown in one or more of formula (I-a) to formula (I-c). The metal corrosion inhibitor disclosure is a multifunctional group metal corrosion inhibitor comprising Schiff base or a derivative structure thereof, a catechol group and a mercapto group. A plurality of active functional groups are introduced, so that the active adsorption site of the inhibitor can be increased, and the synergetic effect of the three functional groups can enhance the adsorption effect of the inhibitor and a metal, And thus, the inhibitor is suitable for applicable for adsorptions of plurality of metals. Therefore, the inhibitor can exert a high anti-corrosion efficiency under the condition of a low addition amount, and provides a protection effect for the plurality of the metals.

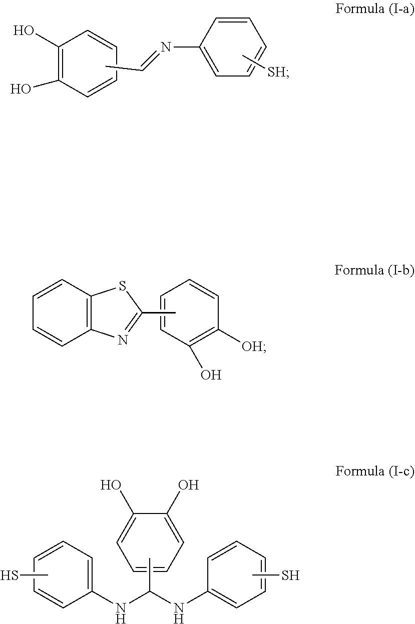

### TECHNICAL FIELD

The present disclosure may be applied in electronic industry and other fields, and particularly, it relates to a multifunctional group metal corrosion inhibitor, a preparation method therefor, and applications thereof in electronic industry and other fields, including but not being limited to, the cleaner for wafer of semiconductor integrated circuit manufacturing.

### BACKGROUND OF THE INVENTION

The cleaner is applied throughout the fields of electronic industry including, but not being limited to production and manufacturing processes of display screens, solar cells, light emitting diodes, printed circuit boards, and semiconductor integrated circuit. However, most of cleaners are corrosive, and when they are applied in the removal of post dry-etched residues, it's easy to corrode metals that incorporated on wafer substrate, thereby resulting in the defects, damage of critical dimension and other problems. To achieve the target function, electronic devices, especially for the semiconductor integrated circuit chips, it experiences a complicated establishment to get the multilayer structure, normally the manufacturing includes some repeating processes like ion implantation, introduction of metals or dielectric layers, photoetching, etching and so on. In this situation, different kinds of metals could be integrated on wafer surface, which makes the corrosion of metal more complex. Due to the difference of metal activity, it tends to cause the electrochemical corrosions rather than regular chemical corrosions. Therefore, it is an important work to develop the cleaner, which is quite challenging to remove all the residues on surface of wafer substrate and protect the metals substrate from the attack of corrosive cleaner in the meantime.

In order to protect the metals substrate and reduce the attacks from corrosive cleaner, the introduction of corrosion inhibitor is a simple and effective way. In general, ammonias, aldehydes and hybrid chemicals that containing N, O, S elements, with physical and chemical adsorptions to metals, can form an adsorption-type protective layer on the surfaces of the metals. The protective layer helps to stop or reduce electrochemical corrosions by isolating the direct contacts of cleaner and metals. However, corrosion inhibitor with single functional group is applied in most of cleaning products. Those products have shortcomings of high cost and low efficiency, because they need high addition amount or synergistic of multiple chemicals to improve anti-corrosion performance with target efficiency and protection ability for different metals.

The efficiency of corrosion inhibitor is related with the numbers and kinds of the active functional groups. Schiff base refers to an organic compound containing characteristic group of an imine or methylenimine, which can be obtained via Kabachnik-Fields reaction of amino and aldehyde groups. Due to the presence of the TT flatness and lone-pair electrons of the nitrogen atom on the imine bond, Schiff base has a better adsorption effect than similar amino- or aldehyde-containing compounds. Therefore, by a molecular design, the multifunctional group inhibitors with Schiff base or a derivative structure thereof may be prepared, which is applied to increase the anti-corrosion efficiency of the inhibitors.

### SUMMARY OF INVENTION

In view of this, the technical problem to be solved by the present disclosure is to provide a multifunctional group metal corrosion inhibitor, a preparation method therefor and applications thereof, wherein the multifunctional group metal corrosion inhibitor can exert a high anti-corrosion efficiency even at a low addition amount.

The present disclosure provides a multifunctional group metal corrosion inhibitor, as shown by one or more of a group consisting of formulas (I-a) to (I-c):

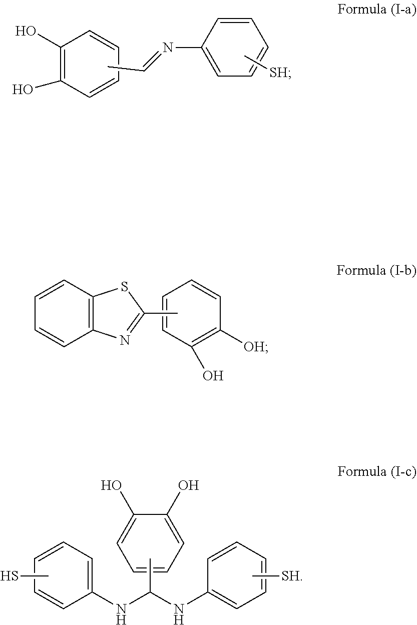

Preferably, it is as shown by one or more of a group consisting of formulas (II-a) to (II-c):

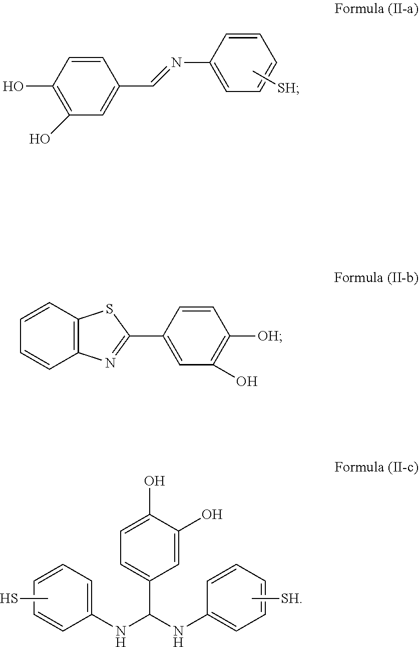

The disclosure further provides a method for preparing a multifunctional group metal corrosion inhibitor, comprising:
reaction between a dihydroxybenzaldehyde of formula (III) and an aminothiophenol of formula (IV) to obtain the multifunctional group metal corrosion inhibitor:

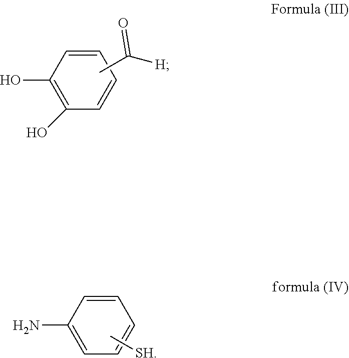

Preferably, the dihydroxybenzaldehyde of formula (III) is selected from a group consisting of 3,4-dihydroxybenzaldehyde and 2,3-dihydroxybenzaldehyde; the aminothiophenol of formula (IV) is one or more selected from a group consisting of 2-aminothiophenol, 3-aminothiophenol and 4-aminothiophenol.

Preferably, a molar ratio of the dihydroxybenzaldehyde of formula (III) to the aminothiophenol of formula (IV) is (0.1 to 10):(0.1 to 10); a temperature of the reaction is 5° C. to 100° C.; a time of the reaction is 0.1 to 48 hours.

Preferably, the reaction is carried out in an organic solvent; the organic solvent is one or more selected from a group consisting of dichloromethane, tetrahydrofuran, acetone, ethanol, methanol and chloroform; a ratio of the dihydroxybenzaldehyde of formula (III) to the organic solvent is 1 g:(5 to 200) mL.

The present disclosure further provides a cleaner comprising the above multifunctional group metal corrosion inhibitor.

Preferably, the cleaner is aqueous; the cleaner comprises water, a multifunctional group metal corrosion inhibitor as claimed in claim 1 or 2 or a multifunctional group metal corrosion inhibitor prepared according to the preparation method of any one of claims 3 to 6, an organic solvent, an etching agent and a buffer system.

Preferably, a mass ratio of the water, the above multifunctional group metal corrosion inhibitor, the organic solvent, the etching agent and the buffer system is (5 to 95):(0.1 to 5):(5 to 95):(0.1 to 5):(0.1 to 10).

Preferably, the organic solvent is one or more selected from a group consisting of N,N-dimethylformamide, sulfolane, dimethyl sulfoxide, 1,4-butyrolactone, diethylene glycol butyl ether, dipropylene glycol butyl ether, tripropylene glycol butyl ether, propylene glycol methyl ether acetate, N, N-dimethylacetamide and N-methylpyrrolidone; the etching agent is one or more selected from a group consisting of ammonium fluoride, hydrofluoric acid and organic amine compounds;
the buffer system is one selected from a group consisting of an ammonium chloride-ammonia system, a Tris-glycine system, a formic acid-ammonium formate system and an acetic acid-ammonium acetate system.

The disclosure provides a multifunctional group metal corrosion inhibitor, as shown by one or more of a group consisting of formula (I-a) to formula (I-c). As compared with the prior art, the metal corrosion inhibitor provided by the present disclosure is a metal corrosion inhibitor with multifunctional groups including Schiff base or derivative structures thereof, catechol and mercapto groups. The designed metal corrosion inhibitor has a plurality of active functional groups introduced, which on one hand, increased the number of the active adsorption sites, and on the other hand, the synergetic effects of the three functional groups can enhance the adsorption effect and ability to different metals, thus leading to a high anti-corrosion efficiency at a low addition amount and provide protection effects for a plurality of the metals.

### DESCRIPTIONS TO THE DRAWINGS

FIG. 1 is a 1H Nuclear Magnetic Resonance Spectra (1H-NMR) of a metal corrosion inhibitor obtained in Example 1 of the present disclosure;

FIG. 2 is a 1H Nuclear Magnetic Resonance Spectra (1H-NMR) of the metal corrosion inhibitor obtained in Example 3 of the present disclosure.

### BEST MODES FOR CARRYING OUT THE INVENTION

With reference to the examples of the present disclosure, the technical solutions in the examples of the present disclosure will be clearly and completely described below, and it is obvious that the described examples are only a part of the examples of the present disclosure, but not all of the examples. Based on the examples in the present disclosure, all the other examples obtained by person skilled in the art without paying any creative efforts belong to the protection scope of the present disclosure.

The disclosure provides a multifunctional group metal corrosion inhibitor, as shown by one or more of a group consisting of formulas (I-a) to (I-c):

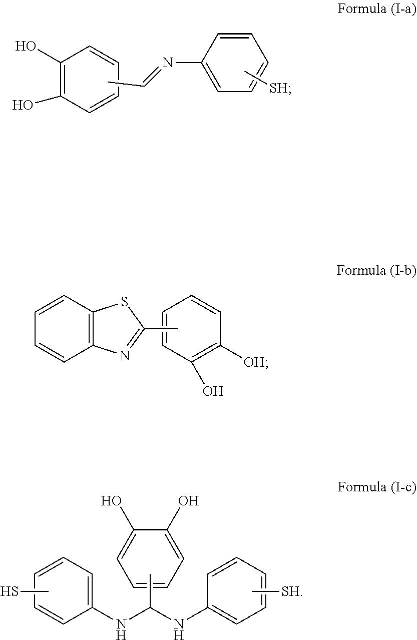

Further preferably, it is as shown by one or more of a group consisting of the following formulas (II-a) to (II-c):

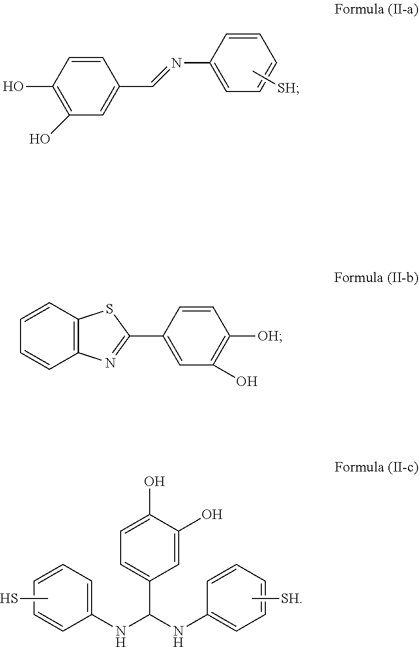

Even further preferably, it is as shown by one or more of a group consisting of the following structures:

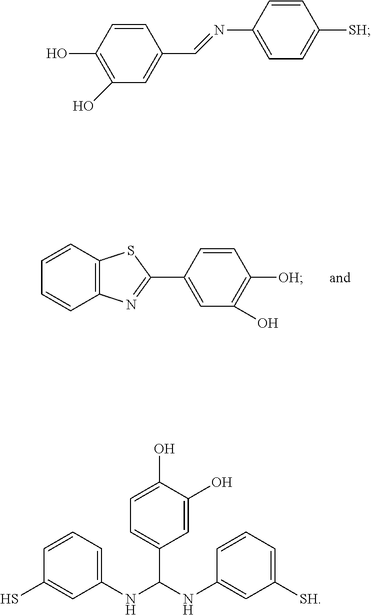 [SMILES: Oc1ccc(/C=N/c2ccc(S)cc2)cc1O ; Oc1ccc(-c2nc3ccccc3s2)cc1O ; Oc1ccc(C(Nc2cccc(S)c2)Nc2cccc(S)c2)cc1O]

The metal corrosion inhibitor provided by the disclosure is a metal corrosion inhibitor with multifunctional groups including Schiff base or a derivative structure thereof, catechol and mercapto groups. The designed metal corrosion inhibitor has a plurality of active functional groups introduced, which on one hand, increased the number of the active adsorption sites, and on the other hand, the synergetic effects of the three functional groups can enhance the adsorption effect and ability to different metals, thus leading to a high anti-corrosion efficiency at a low addition amount and provide protection effects for a plurality of the metals.

The present disclosure further provides a method for preparing the above multifunctional group metal corrosion inhibitor, comprising: reaction between a dihydroxybenzaldehyde of formula (III) and an amino thiophenol of formula (IV) to obtain a multifunctional group metal corrosion inhibitor:

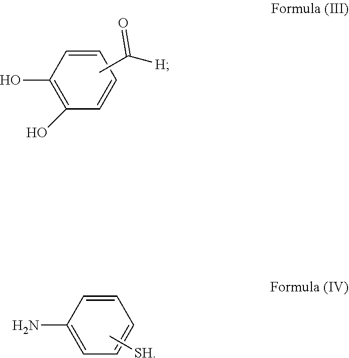

In the present disclosure, the dihydroxybenzaldehyde of formula (III) is preferably 3,4-dihydroxybenzaldehyde; the aminothiophenol of formula (IV) is preferably one or more of 2-aminothiophenol, 3-aminothiophenol and 4-aminothiophenol; a molar ratio of the dihydroxybenzaldehyde of formula (III) to the aminothiophenol of formula (IV) is (0.1 to 10):(0.1 to 10), more preferably 1:(1 to 5), further preferably 1:(1 to 3), still further preferably 1:(1 to 2.5), and most preferably 1:(1 to 2.2); in the examples provided by the present disclosure, the molar ratio of the dihydroxybenzaldehyde of formula (III) to the aminothiophenol of formula (IV) is particularly 1:1.1, 1:2.2 or 1:1.

In the present disclosure, the reaction is preferably carried out in an organic solvent; the organic solvent is preferably one or more of a group consisting of dichloromethane, tetrahydrofuran, acetone, ethanol, methanol and chloroform; a ratio of the dihydroxybenzaldehyde of formula (III) to the organic solvent is 1 g:(5 to 200) mL, more preferably 1 g:(10 to 100) mL, further preferably 1 g:(20 to 80) mL, still further preferably 1 g:(10 to 50) mL, and most preferably 1 g:(30 to 40) mL.

The present disclosure, without any particular limitations on the order of adding the above raw materials, may directly mix the dihydroxybenzaldehyde of formula (III) and the aminothiophenol of formula (IV) in the organic solvent, or dissolve the dihydroxybenzaldehyde of formula (III) and the aminothiophenol of formula (IV) in the organic solvent respectively and then mix them; the mixing method may be either a direct mixing or selected to be a dropwise addition, without any particular limitations.

In the present disclosure, a temperature of the reaction is preferably 5° C. to 100° C., more preferably 20° C. to 80° C., and further preferably 25° C. to 60° C.; A time of the reaction is preferably 0.1 to 48 hours, more preferably 1 to 40 hours, further preferably 5 to 30 hours, yet further preferably 5 to 20 hours, still further preferably 6 to 10 hours, and most preferably 8 hours.

After the reaction is finished, the organic solvent and unreacted raw materials may be directly removed, and the multifunctional group metal corrosion inhibitor may be obtained; also, after the reaction, the reaction mixture may be kept standing at room temperature to precipitate a solid, which is the multifunctional group metal corrosion inhibitor; further, a second organic solvent may be used for recrystallization to obtain the multifunctional group metal corrosion inhibitor; the second organic solvent is preferably one or more of a group consisting of dichloromethane, methanol, ethanol and chloroform.

The resultant multifunctional group metal corrosion inhibitor is also preferably dried; the drying is preferably vacuum drying; a temperature of the drying is preferably 40° C.-60° C., more preferably 50° C.; a time of the drying is preferably 2-10 hours, more preferably 4-8 hours, and further preferably 6 hours.

According to the present disclosure, a plurality of active functional groups are introduced by selecting the raw materials. On one hand, the increasing of active adsorption sites effectively inhibit the corrosions of metals even at a low addition amount of corrosion inhibitors; on the other hand, the metal corrosion inhibitor is prepared by the reaction between amino and aldehyde containing compounds, which endows the corrosion inhibitor with Schiff base structure or its derivatives, further through its synergetic effects with catechol and mercapto groups, can enhance the adsorption effect of the inhibitor on metals.

The disclosure also provides a cleaner comprising the above multifunctional group metal corrosion inhibitor.

In the present disclosure, the cleaner is preferably aqueous.

In the present disclosure, the cleaner preferably comprises water, the multifunctional group metal corrosion inhibitor, an organic solvent, an etching agent and a buffer system; a mass ratio of the water, the multifunctional group metal corrosion inhibitor, the organic solvent, the etching agent and the buffer system is preferably (5 to 95):(0.1 to 5):(5 to 95):(0.1 to 5):(0.1 to 10), more preferably (10 to 90):(0.1 to 5):(10 to 90):(0.1 to 5):(0.1 to 10), further preferably (30 to 70):(0.1 to 5):(30 to 70):(0.1 to 5):(0.1 to 10), yet further preferably (40 to 60):(0.1 to 5):(40 to 60):(0.1 to 5):(0.1 to 10), still further preferably (40 to 60):(0.5 to 3):(40 to 60):(0.1 to 3):(2 to 10), yet still further preferably (40 to 60):(0.5 to 2):(40 to 60):(0.5 to 1.5):(2 to 8), and most preferably (50 to 60):(0.5 to 1.5):(50 to 60):(0.5 to 1):(3 to 6).

In the present disclosure, the organic solvent is preferably one or more of a group consisting of N,N-dimethylformamide, sulfolane, dimethyl sulfoxide, 1,4-butyrolactone, diethylene glycol butyl ether, dipropylene glycol butyl ether, tripropylene glycol butyl ether, propylene glycol methyl ether acetate, N,N-dimethylacetamide, and N-methylpyrrolidone.

The etching agent is preferably one or more of a group consisting of ammonium fluoride, hydrofluoric acid and organic amine compounds; the organic amine compounds may be at least one of a group consisting of primary amines, secondary amines, tertiary amines, ammoniums and other organic compounds, preferably at least one of a group consisting of methylamine, ethylamine, hydroxylamine, octylamine, triethylamine, acetylamide, diethylamine, tert-butylamine, butyrylamide, dopamine, isobutylamine, isopentylamine, n-propylamine, n-hexylamine, cyclopropylamine, cyclohexylamine, cycloheptylamine, cyclopentylamine, heptylamine, ethanolamine, diethanolamine, diglycolamine, isopropanolamine, triisopropanolamine, diisopropanolamine, N-ethylethanolamine, N-phenylethanolamine, N-acetylethanolamine, N-butyldiethanolamine, N-cyclohexylethanolamine, N-benzyldiethanolamine, N-phenyldiethanolamine, N-dibenzylethanolamine, N-tert-butyldiethanolamine, N-tert-butylisopropanolamine, N-methyldiisopropanolamine, 1,6-hexamethylenediamine, 1,2-propylenediamine, 1,3-propylenediamine, 1,4-butylenediamine, 1,8-octanediamine, triethylenediamine, diethylenetriamine, adipamide, 2-Butenediamide, tri-n-dodecylamine, 2-octyldodecylamine, N-ethylethylenediamine, N-methylethylenediamine, N-benzylethylenediamine, N-phenylethylenediamine, spermine, N-acetylethylenediamine, pentaethylenehexamine, tetraethylenepentamine, 1H-pyrazole-3,5-diamine, 3-diethylaminopropylamine, 3-dimethylaminopropylamine, N-butylethylenediamine, N-isopropylethylenediamine, N-methyl-p-phenylenediamine, tetramethylmethanediamine, tetrabutylammonium hydroxide, tetraethylammonium hydroxide, tetrapropylammonium hydroxide, benzyltrimethylammonium hydroxide, N-(benzylcarbonyloxy) hydroxylamine, and N,N-dibenzylhydroxylamine.

The buffer system is one selected from an ammonium chloride-ammonia system, a Tris-glycine system, a formic acid-ammonium formate system and an acetic acid-ammonium acetate system; a mass ratio of the ammonium chloride to the ammonia is preferably (1.5 to 3):(1.5 to 3); a mass ratio of the Tris to the glycine is preferably (1.5 to 3):(1.5 to 3); a mass ratio of the formic acid to the ammonium formate is preferably (1.5 to 3):(1.5 to 3); a mass ratio of the acetic acid to the ammonium acetate is preferably (1.5 to 3):(1.5 to 3).

In the present disclosure, the cleaner, in addition to the above components, may further comprise one or more of a group consisting of inorganic acids, alcohol compounds, surfactants and antioxidants; they can be selectively added according to cleaning objects and cleaning requirements.

The inorganic acids are well known for a person skilled in the art, without any particular limitations. In the present disclosure, preferences are given to one or more of a group consisting of sulfuric acid, nitric acid, hydrochloric acid, phosphoric acid, boric acid, and carbonic acid.

The alcohol compounds are well known for a person skilled in the art, without any particular limitations. In the present disclosure, preferences are given to one or more of a group consisting of ethylene glycol, glycerol, 1,3-butanediol, 1,2-pentanediol, pentaerythritol, 2,5-hexanediol, polycaprolactone diol, 1,6-hexanediol, sorbitol, neopentyl glycol, 1,4-butanediol, 1,2,6-hexanetriol, xylitol, L-mannitol, D (+)-arabitol, geraniol, dulcitol, 1,2,4-butanetriol, furfuryl alcohol, phytol, dipentaerythritol, L-threoninol, L-talitol, glucose, erythritol, and xylose.

The surfactants are well known for a person skilled in the art, without any particular limitations. In the present disclosure, preferences are given to one or more of a group consisting of 3,5-dimethyl-1-hexyn-3-ol, lauryl alcohol ether phosphate, cocinic acid diethanolamide, cocoamidopropyl betaine, cocoamidopropyl hydroxy sulfobetaine, lauramidopropyl hydroxyl sulfobetaine, acetoxime, lauramidopropyl amine oxide, monoglyceride, Span, and polyoxy-15 hydroxystearate.

The antioxidants are well known for a person skilled in the art, without any particular limitations. In the present disclosure, preferences are given to one or more of a group consisting of butylhydroxyanisole, 2,6-di-t-butyl-p-cresol, hydroquinone, D-isoascorbic acid, sorbitol, phytic acid, chitosan, and chitosan oligosaccharide.

The disclosure further provides an application of the above multifunctional group metal corrosion inhibitor in electronic industry and other fields; the applications in electronic industry and other fields include, but are not limited to, cleaner used for display screens, solar cells, light emitting diodes, printed circuit boards, and semiconductor integrated circuits, without any particular limitations.

The disclosure also provides an application of the above multifunctional group metal corrosion inhibitor in cleaner for semiconductor wafer.

The disclosure further provides an application of the above cleaner for semiconductor wafer.

To further illustrate the present disclosure, a multifunctional group metal corrosion inhibitor provided by the present disclosure, a preparation method therefor and applications thereof are described below in detail with the reference to the examples.

All the reagents used in the following examples are commercially available.

### EXAMPLE 1

To a round-bottom flask, 0.824 g (6.58 mmol) of 3,4-dihydroxybenzaldehyde, 1 g (7.24 mmol) of 2-aminothiophenol and 30 mL of ethanol were added, and after reacting for 6 hours at 60° C., the reaction mixture was kept standing at room temperature for 12 hours until a yellow solid was precipitated. After filtration, the obtained solid was oven dried, to produce a metal corrosion inhibitor, labelled as ATOH-1.

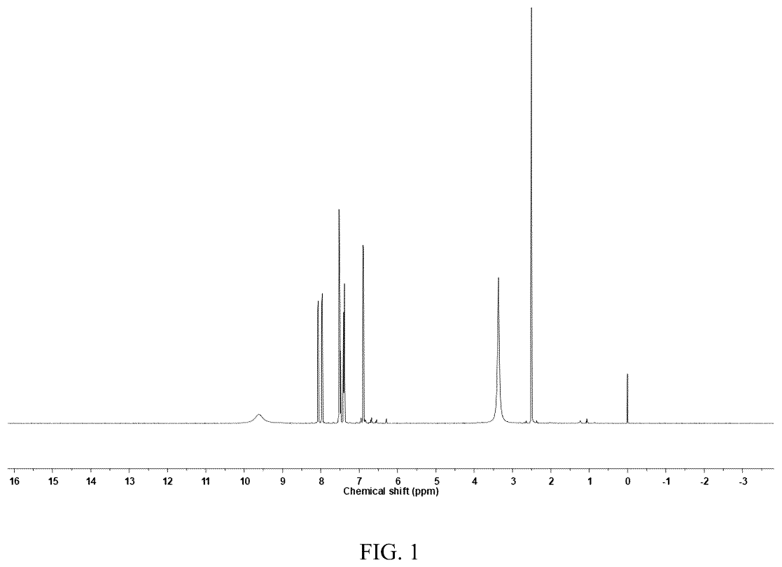

The metal corrosion inhibitor obtained in Example 1 was analyzed by nuclear magnetic resonance, to obtain its 1H nuclear magnetic resonance spectrum, as shown in FIG. 1.

### EXAMPLE 2

To a round-bottom flask, 0.824 g (6.58 mmol) of 3,4-dihydroxybenzaldehyde, 1 g (7.24 mmol) of 2-aminothiophenol and 30 mL of ethanol were added, and after reacting for 12 hours at 35° C., the reaction mixture was kept standing at room temperature for 12 hours until a yellow solid was precipitated. After filtration, the obtained solid was oven dried, to produce a metal corrosion inhibitor, labelled as ATOH-2.

The metal corrosion inhibitor obtained in Example 2 was analyzed by nuclear magnetic resonance, and its nuclear magnetic resonance structure was similar to that of Example 1. It may be concluded that the two products were in the same structure.

### COMPARATIVE EXAMPLE 1

To a round-bottom flask, 0.698 g (6.58 mmol) of benzaldehyde, 1 g (7.24 mmol) of 2-aminothiophenol and 30 mL of ethanol were added, and after reacting for 6 hours at 60° C., the reaction mixture was kept standing at room temperature for 12 hours until a yellow solid was precipitated. After filtration, the obtained solid was oven dried, to produce a product, labelled as C1.

### COMPARATIVE EXAMPLE 2

To a round-bottomed flask, 0.803 g (6.58 mmol) of m-hydroxybenzaldehyde, 1 g (7.24 mmol) of 2-aminothiophenol and 30 mL of ethanol were added, and after reacting for 6 hours at 60° C., the reaction mixture was kept standing at room temperature for 12 hours until a yellow solid was precipitated. After filtration, the obtained solid was oven dried, to produce a product, labelled as C2.

The metal corrosion inhibitors obtained in Examples 1-2 and the products obtained in Comparative Examples 1-2 were applied in the same kind of cleaner to detect their performances, wherein the mass ratio of N,N-dimethylacetamide, water, ammonium fluoride and a buffer system in the compositions was 7.5:11.25:0.1:0.93; the buffer system was an acetic acid-ammonium acetate system with a mass ratio of 2:2.7; 1.1 wt % of the metal corrosion inhibitors or products of the comparative examples thereof were added into the compositions, and under equivalent conditions, a four-probe method was used to test metal corrosion rates, and the constitutions and performance testing results of cleaner were shown in Table 1.

TABLE 1
The constitutions and performance testing results of cleaner

| | | Comparative Example | Comparative Example |
|---|---|---|---|
| Items | Examples 1, 2 | 1 | 2 |
| Corrosion inhibitors | 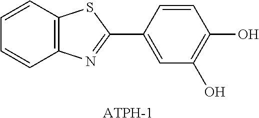 | 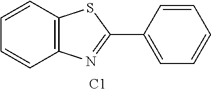 | 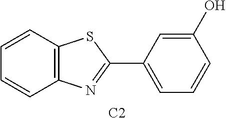 |
| Al corrosion rate- | 2.1 Å/min | 5.6 Å/min | 3 Å/min |
| 40° C. | | | |
| Cu corrosion rates- | 0.1 Å/min | 0.4 Å/min | 3.7 Åmin |
| 40° C. | | | |

### EXAMPLE 3

To a round-bottom flask, 1 g (7.24 mmol) of 3,4-dihydroxybenzaldehyde, 1.99 g of 3-aminothiophenol (15.93 mmol) and 30 mL of ethanol were added, and after reacting for 8 hours at 60° C., the reaction mixture was kept standing at room temperature for 12 hours until a yellow solid was precipitated. After filtration, the obtained solid was oven dried, to produce a metal corrosion inhibitor, labelled as ATOH-3.

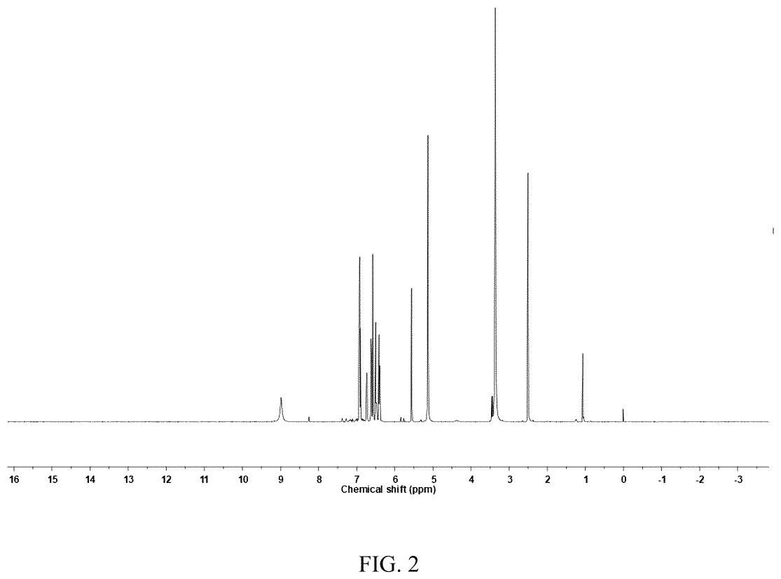

The metal corrosion inhibitor obtained in Example 3 was analyzed by nuclear magnetic resonance, to obtain its 1H nuclear magnetic resonance spectrum, as shown in FIG. 2.

### COMPARATIVE EXAMPLE 3

To a round-bottom flask, 1 g (9.42 mmol) of benzaldehyde, 2.59 g (20.73 mmol) of 3-aminothiophenol and 30 mL of ethanol were added, and after reacting for 8 hours at 60° C., the reaction mixture was kept to stand at room temperature for 12 hours until a yellow solid was precipitated. After filtration, the obtained solid was oven dried, to produce a compound C3.

### COMPARATIVE EXAMPLE 4

To a round-bottom flask, 1 g (8.19 mmol) of m-hydroxybenzaldehyde, 2.26 g (18.02 mmol) of 3-aminothiophenol and 30 mL of ethanol were added, and after reacting for 8 hours at 60° C., the reaction mixture was kept standing at room temperature for 12 hours until a yellow solid was precipitated. After filtration, the obtained solid was oven dried, to produce a compound C4.

### COMPARATIVE EXAMPLE 5

To a round-bottom flask, 1 g (7.24 mmol) of 3,4-dihydroxybenzaldehyde, 1.48 g (15.93 mmol) of aniline and 30 mL of ethanol were added, and after reacting for 8 hours at 60° C., the reaction mixture was kept standing at room temperature for 12 hours until a yellow solid was precipitated. After filtration, the obtained solid was oven dried, to produce a compound C5.

The metal corrosion inhibitor obtained in Example 3 and the products obtained in Comparative Examples 3-5 were applied in the same kind of cleaner to detect their performances, wherein the mass ratio of N,N-dimethylacetamide, water, ammonium fluoride and a buffer system in the compositions was 7.5:11.25:0.1:0.93; the buffer system is an acetic acid-ammonium acetate system with a mass ratio of 2:2.7; 1.1 wt % of the metal corrosion inhibitors or products of the comparative examples thereof were added into the compositions, and under equivalent conditions, a four-probe method was used to test metal corrosion rates, and the constitutions and performance testing results of cleaner were shown in Table 2.

TABLE 2
The constitutions and performance testing results of cleaner

| | | | Comparative |
|---|---|---|---|
| Items | | Example 3 | Example 3 |
| Corrosion inhibitors | | 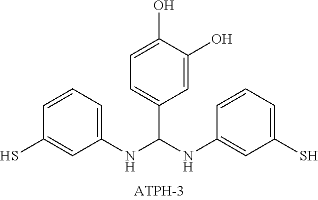 [SMILES: Oc1ccc(C(Nc2cccc(S)c2)Nc2cccc(S)c2)cc1O] | 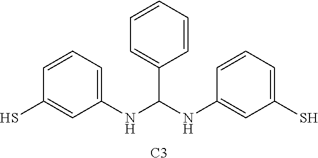 |
| Al corrosion rate- | | 1.1 Å/min | 2.4 Å/min |
| 40° C. | | | |
| Cu corrosion | | 0.2 Å/min | 1.3 Å/min |
| rates- 40° C. | | | |
| | | Comparative | Comparative |
| | Items | Example 4 | Example 5 |
| | Corrosion inhibitors | 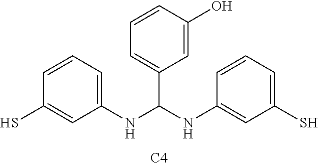 | 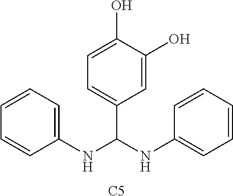 |
| | Al corrosion rate- | 2.5 Å/min | 6.5 Å/min |
| | 40° C. | | |
| | Cu corrosion | 4.5 Å/min | 2.7 Å/min |
| | rates- 40° C. | | |

### EXAMPLE 4

To a round-bottom flask, 1 g (7.24 mmol) of 3,4-dihydroxybenzaldehyde and 20 ml of ethanol were added, and slowly dropwise added with 10 ml of an ethanol solution dissolved with 0.91 g (7.24 mmol) of 4-aminothiophenol, and after reacting for 8 hours at room temperature, the organic solvent was removed by rotary evaporation. The precipitated solid was oven dried, to produce a metal corrosion inhibitor, labelled as ATOH-4.

### COMPARATIVE EXAMPLE 6

To a round-bottom flask, 1 g (7.24 mmol) of 3,4-dihydroxybenzaldehyde and 20 ml of ethanol were added, and slowly dropwise added with 10 ml of an ethanol solution dissolved with 0.67 g (7.24 mmol) of aniline, and after reacting for 8 hours at room temperature, the organic solvent was removed by rotary evaporation. The precipitated solid was oven dried, to produce a compound C6.

### COMPARATIVE EXAMPLE 7

To a round-bottom flask, 1 g (8.19 mmol) of p-hydroxybenzaldehyde and 20 ml of ethanol were added, and slowly dropwise added with 10 ml of an ethanol solution dissolved with 1.03 g (8.19 mmol) of 4-aminothiophenol, and after reacting for 8 hours at room temperature, the organic solvent was removed by rotary evaporation. The precipitated solid was oven dried, to produce a compound C7.

### COMPARATIVE EXAMPLE 8

To a round-bottom flask, 1 g (9.42 mmol) of benzaldehyde and 20 mL of ethanol were added, and slowly dropwise added with 10 mL of an ethanol solution dissolved with 1.18 g (9.42 mmol) of 4-aminothiophenol, and after reacting for 8 hours at room temperature, the organic solvent was removed by rotary evaporation. The precipitated solid was oven dried, to produce a compound C8.

The metal corrosion inhibitor obtained in Example 4 and the products obtained in Comparative Examples 6-8 were applied in the same kind of cleaner to detect their performances, wherein the mass ratio of N,N-dimethylacetamide, water, ammonium fluoride and a buffer system in the compositions was 7.5:11.25:0.1:0.93; the buffer system is an acetic acid-ammonium acetate system with a mass ratio of 2:2.7; 1.1 wt % of the metal corrosion inhibitors or products of the comparative examples thereof were added into the compositions, and under equivalent conditions, a four-probe method was used to test metal corrosion rates, and the constitutions and performance testing results of cleaner were shown in Table 3.

TABLE 3
The constitutions and performance testing results of cleaner

| | | Comparative |
|---|---|---|
| Items | Example 4 | Example 6 |
| Corrosion inhibitors | 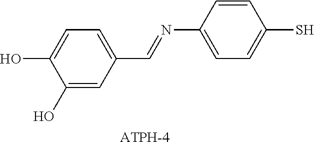 [SMILES: Oc1ccc(/C=N/c2ccc(S)cc2)cc1O ; "ATPH-4"] | 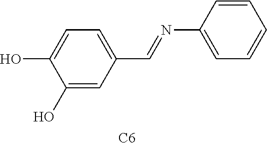 |
| Al corrosion | 1.2 Å/min | 10.6 Å/min |
| rate- 40° C. | | |
| Cu corrosion | >0.1 Å/min | 1 Å/min |
| rates- 40° C. | | |
| | Comparative | Comparative |
| Items | Example 7 | Example 8 |
| Corrosion inhibitors | 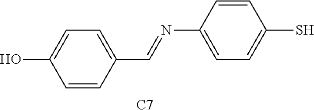 | 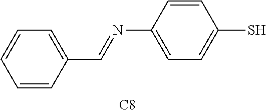 |
| Al corrosion rate- | 1.9 Å/min | 6.5 Å/min |
| 40° C. | | |
| Cu corrosion | 5.5 Å/min | 2.2 Å/min |
| rates- 40° C. | | |

As can be seen from Tables 1 to 3, the anti-corrosion effects of Examples 1 to 3 on metals each are far better than those of Comparative Examples 1 to 8, and this demonstrates that Schiff base or a derivative group thereof, catechol and mercapto groups have synergetic effects so that the metal corrosion inhibitors obtained in the present disclosure have excellent anti-corrosion effects. However, the compounds prepared in Comparative Examples 1-8 have poorer anti-corrosion effects because they do not simultaneously contain the above three functional groups.

1. A multifunctional group metal corrosion inhibitor, as shown by one or more of a group consisting of formulas (I-a) to (I-c):

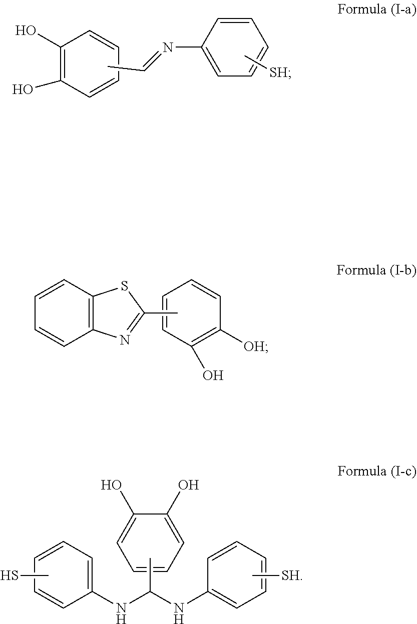

2. The multifunctional group metal corrosion inhibitor according to claim 1, as shown by one or more of a group consisting of formulas (II-a) to (II-c):

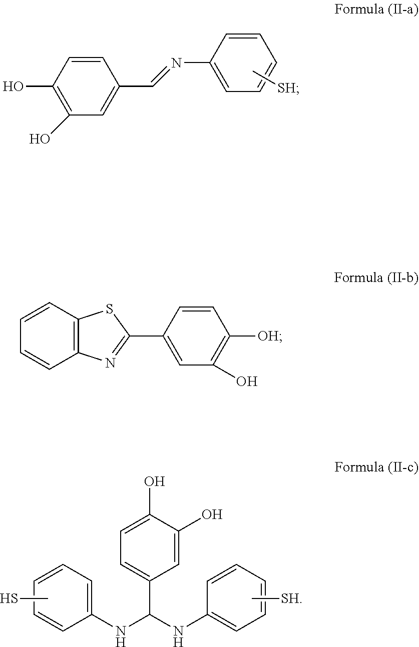

3. A method for preparing a multifunctional group metal corrosion inhibitor, the method comprising:
reaction between a dihydroxybenzaldehyde of formula (III) and an aminothiophenol of formula (IV) to obtain the multifunctional group metal corrosion inhibitor:

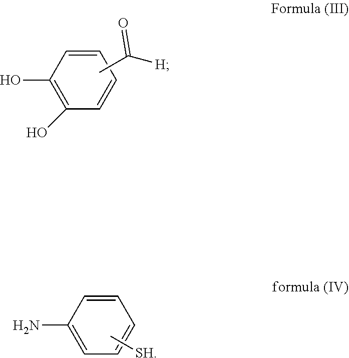

4. The method according to claim 3, wherein the dihydroxybenzaldehyde of formula (III) is selected from 3,4-dihydroxybenzaldehyde and 2,3-dihydroxybenzaldehyde; and the aminothiophenol of formula (IV) is one or more selected from a group consisting of 2-aminothiophenol, 3-aminothiophenol and 4-aminothiophenol.

5. The method according to claim 3, wherein a molar ratio of the dihydroxybenzaldehyde of formula (III) to the aminothiophenol of formula (IV) is (0.1 to 10)_:(0.1 to 10); a temperature of the reaction is 5° C. to 100° C.; and a time of the reaction is 0.1 to 48 hours.

6. The method according to claim 3, wherein the reaction is carried out in an organic solvent; the organic solvent is one or more selected from a group consisting of dichloromethane, tetrahydrofuran, acetone, ethanol, methanol and chloroform; and a ratio of the dihydroxybenzaldehyde of formula (III) to the organic solvent is 1 g:(5 to 200) mL.

7. A cleaner comprising the multifunctional group metal corrosion inhibitor of claim 1.

8. The cleaner according to claim 7, wherein the cleaner is aqueous; and the cleaner comprises water, the multifunctional group metal corrosion inhibitor, an organic solvent, an etching agent and a buffer system.

9. The cleaner according to claim 8, wherein a mass ratio of the water, the multifunctional group metal corrosion inhibitor, the organic solvent, the etching agent and the buffer system is (5 to 95):(0.1 to 5):(5 to 95):(0.1 to 5):(0.1 to 10).

10. The cleaner according to claim 8, wherein:
the organic solvent is one or more selected from a group consisting of N,N-dimethylformamide, sulfolane, dimethyl sulfoxide, 1,4-butyrolactone, diethylene glycol butyl ether, dipropylene glycol butyl ether, tripropylene glycol butyl ether, propylene glycol methyl ether acetate, N,N-dimethylacetamide and N-methylpyrrolidone;
the etching agent is one or more selected from a group consisting of ammonium fluoride, hydrofluoric acid and organic amine compounds; and
the buffer system is one selected from a group consisting of an ammonium chloride-ammonia system, a Tris-glycine system, a formic acid-ammonium formate system and an acetic acid-ammonium acetate system.

11. A cleaner comprising the multifunctional group metal corrosion inhibitor of claim 2.

12. A cleaner comprising the multifunctional group metal corrosion inhibitor prepared according to the method of claim 3.

13. A cleaner comprising the multifunctional group metal corrosion inhibitor prepared according to the method of claim 4.

14. A cleaner comprising the multifunctional group metal corrosion inhibitor prepared according to the method of claim 5.

15. A cleaner comprising the multifunctional group metal corrosion inhibitor prepared according to the method of claim 6.
